(19) United States
(12) Patent Application Publication
(10) Pub. No.: US 2022/0339042 A1
(43) Pub. Date: Oct. 27, 2022

(54) Bandage System with Decongestive Function

(71) Applicants: Dr. Recknagel Gesundheitsservice GmbH, Berlin (DE); GPB Gesundheitszentrum Prenzlauer Berg GmbH, Berlin (DE)

(72) Inventor: Uwe Valder, Ahlsdorf (DE)

(21) Appl. No.: 17/762,379

(22) PCT Filed: Sep. 25, 2019
(86) PCT No.: PCT/DE2019/100847

§ 371 Date: Mar. 22, 2022

(51) Int. Cl. A61F 13/08; A61F 13/00

(52) U.S. Cl. CPC A61F 13/085; A61F 13/00021; A61F 13/00038

(57) ABSTRACT

A bandage system with decongestion function for the treatment of disorders of the lymphatic and/or venous system of the human body, consisting of a fabric which has alternately strip-shaped regions with relative extensibility and without extensibility and at least partially encases the human body and/or a limb.

According to the invention
the extension direction of the fabric regions of relative extensibility (9) is oriented longitudinally to the working direction of the musculature of the body or of the limbs,
the fabric regions without extensibility (10) run transversely to the working direction of the musculature,
the fabric regions of relative extensibility (9) and the fabric regions without extensibility (10) are arranged successively alternately in working direction of the musculature of the body or of the limbs and
the bandage system is adjustable in its circumference.

### CROSS REFERENCE TO RELATED APPLICATIONS

This application is the U.S. national stage of International Application No. PCT/DE2019/100847, filed on 2019 Sep. 25.

### BACKGROUND

The invention proceeds from a bandage system with decongestion function for the treatment of disorders of the lymphatic and/or venous system of the human body according to the type of the introductory clause of claim 1.

Bandage systems, also named as orthoses, are known in a wide variety of embodiments and for the most diverse applications. They are used in the case of venous diseases and/or disorders of the lymphatic system, so-called oedemas, and as prophylaxis of disorders of the venous and/or lymphatic system in the resting body and in the case of limbs in an inactive lying position, for example as adjunctive therapy after operations.

For example, a therapeutic stocking is known, which exerts a pressing force against the legs with a pressure profile which decreases from the ankle to the upper thigh (DE 39 16 447 A1).

Furthermore, a compressive orthosis is known, in particular a compression stocking which has at least one tube-like base body made from an elastically yielding base material which is formed isotropically with constrictions running continuously in longitudinal direction. Longitudinal ribs and, if applicable, transverse ribs, run between the constrictions (DE 20 2006 006 884 U1).

In another leg stocking for compression therapy, the fabric consists of a nubbed flat knit, which produces a combined compression-massage effect (DE 20 2008 017 054 U1).

A compression sleeve is also known for the human-medical treatment of the varicose symptom complex of the human legs, which consists of a strip of a relatively thin highly elastic material. At its abutment—or respectively overlapping site, the strip is closed in a variable manner by adjustable closures, for example hook-and-loop tapes, whereby the compression pressure is able to be adapted to the individual anatomy of the human leg and to the requirements of the individually pronounced disease areas (DE 42 30 165 A1).

The effect of all these therapeutic stockings is based on the fabric which is used exerting a compression force onto the body or respectively onto the limbs. The exerting of compression forces is, however, not optimally effective for preventing and/or reducing the formation of oedema. A further disadvantage of these orthoses consists in the low acceptance by the patients. The reasons lie both in the laborious procedure of putting them on and also in the high physical stress and the discomfort when being worn. As the material which is used must have a particular inherent elasticity, elastic fibres with a high proportion of synthetic fibres are generally used, which can lead to discomfort for people who have sensitive skin, and can not be used at all for people who have a total skin incompatibility with synthetic fabrics.

To facilitate putting on a tubular bandage, without its therapeutic effect being lost after its being applied, it is known to produce the bandage from elastic and substantially less elastic parts. In a first embodiment, the bandage consisting essentially of a less elastic part is provided with a longitudinal strip of a material with higher elasticity, which is bridged by means of bands which are non-elastic in longitudinal direction and are fastened to the non-elastic part so that its elasticity can be at least limited. The free end of the non-elastic bands is drawn by a loop fastened to the non-elastic part on the opposite side and is able to be fixed to itself for example by means of a hook-and-loop closure. Thereby, the effective width of the elastic strip can be changed. The elastic longitudinal strip can be woven into or worked into the part with the lower elasticity.

In a second embodiment, a part with low elasticity and a part with high elasticity are arranged respectively in pairs alternately, lying opposite one another. Respectively the parts with the high elasticity are bridged by non-elastic bands which are fastened in an identical manner as in the above-mentioned first variant on the part with the lower elasticity (EP 0762 858 B1). This bandage is provided only for the support of joints. An application for the prophylaxis or treatment of disorders of the venous and/or lymphatic system by decongestion is not provided. In the case of an application of this bandage for such a treatment, the same disadvantages would occur as in the bandage systems described above, as it exerts an undifferentiated compression force onto the body or onto the limbs.

Furthermore, a variety of compression garments, functional stockings, compression orthoses and compression aids are known, which have zones with different extensibility and/or pressure gradients. These zones of different extensibility and/or pressure gradients extend in circumferential direction of the limbs and surround these completely (WO 2017213792 A1, AU 2013237693 B2, EP 1885310 B1, EP 2348902 B1, EP 0 927 014 B1). In individual cases they are embodied in a divided manner in longitudinal direction of the limbs and are provided with adjustable closures (AU 2013237693 B2). The effect of these orthoses likewise consists in the exerting of compression force for decongesting the oedemas or respectively for preventing oedema formation, which, however, generally does not, but at least not entirely, provide the desired decongestion, result in a restriction in mobility through the high physical stress and are laborious to put on and take off.

### SUMMARY

The invention proceeds from a bandage system with decongestion function for the treatment of disorders of the lymphatic and/or venous system of the human body, consisting of a fabric which has alternately strip-shaped regions with relative extensibility and without extensibility and at least partially encases the human body and/or a limb.

According to the invention
the extension direction of the fabric regions of relative extensibility (9) is oriented longitudinally to the working direction of the musculature of the body or of the limbs,
the fabric regions without extensibility (10) run transversely to the working direction of the musculature,
the fabric regions of relative extensibility (9) and the fabric regions without extensibility (10) are arranged successively alternately in working direction of the musculature of the body or of the limbs and
the bandage system is adjustable in its circumference.

The bandage system according to the invention enables a highly effective decongestion function, without the fabric of the bandage system having to apply for this a compression force onto the human body or the limbs.

### DETAILED DESCRIPTION

The invention is therefore based on the problem of developing a bandage system, the decongestion function of which has a high efficacy, both with regard to a reduction of existing oedemas and also with regard to prophylaxis, therefore the proactive prevention of disorders of the venous and/or lymphatic system. In addition, the bandage system is to have a high degree of comfort when being worn and to be able to be handled easily.

The problem is solved according to the invention by the characterizing features of claim 1.

The bandage system according to the invention enables a highly effective decongestion function, without the fabric of the bandage system having to apply a compression force onto the human body or onto the limbs for this. This compressionless decongestion is achieved in that the extending direction of the fabric regions of relative extensibility runs longitudinally to the working direction of the musculature of the body or of the limbs and therefore transversely to the split line course of the skin. Thereby, the bandage system operates in a directionally oriented manner.

By comparison, the non-extensible fabric regions run approximately in the direction of the split lines of the skin, i.e. transversely to the longitudinal direction of the extremity or respectively working direction of the musculature. The fabric regions with and without extensibility are arranged successively in an alternating manner in the working direction of the musculature of the body or of the limbs, so that a particular number of strip-shaped fabric regions are arranged alternately with and without extensibility over the length of the bandage system. The width of the non-extensible fabric regions is adapted individually to the patient. It conforms to the swellings of the body part. The greater the swelling, the wider the non-extensible fabric region is designed.

The bandage system is adjustable in its circumference for adaptation of the bandage system to different fatnesses of bodies and/or limbs and to receding oedemas. This is achieved in that the non-extensible fabric regions of the bandage system, running in an annular manner, are not closed continuously, therefore do not run around the body or the extremity over the full circumference. The interruption of the non-extensible fabric regions is situated in bandage systems for extremities advantageously on the inner side thereof, and in bandage systems for the torso on the front side thereof. In these interrupted fabric regions, to guarantee the secure abutting of the bandage system against the body or respectively body part, an adjustable and/or re-adjustable closure system, e.g. a hook-and-loop system, is provided. Thereby, the bandage system can be tightened in the case of receding oedemas and can thus be adapted to the changing body shape. The extensible fabric can, however, form entirely a closed casing in this region, so that the adjustable region can therefore be provided with the same fabric which is extensible in the working direction of the musculature of the body or of the limbs. The extensible fabric regions of the bandage system therefore form in the case as a whole a closed envelope of the body or of the extremity.

As already mentioned above, no compression force originates from the fabric of the bandage system according to the invention onto the skin of the body or of the limbs. The bandage system fulfils here the task of the known compression stockings, namely of supporting the venous system, in order to minimize a retaining of tissue water, however, it fulfils this task without, in so doing, impairing the working of the superficial lymphatic system. The fabric of the bandage system according to the invention thereby replaces the biomechanical characteristics of the skin, the extending of which is likewise different in the two directions running perpendicularly to one another. In the direction of its split line, therefore transversely to the longitudinal direction of the extremities and hence to the working direction of the musculature, the skin is substantially less extensible and thus has a higher tension in this direction than in the working direction of the musculature, in which, in turn, the tension is distinctly less owing to the greater extensibility.

These medically-anatomically acting advantages also provide, however, substantial advantages with regard to comfort when wearing the new bandage system. As already explained above, through the compressionless decongestion, the fabric of the bandage system exerts no, but at least no noticeable compression forces onto the human body or the limbs. Through the distinctly reduced physical stress and smaller restriction of the mobility of the body areas and limbs encased by the bandage system associated therewith, the comfort when wearing the bandage system increases considerably. As the production of the extensibility is not connected to particular synthetic materials, a better skin compatibility is also provided in the use of natural fibres for the production of the fabrics, such as wool, linen, amongst others. In addition, the production of the fabrics from such materials for the bandage system according to the invention is more economical, because the fibres do not have to possess any inherent elasticity, so that no particular requirements have to be imposed on the machines which are used for the production of the fabrics, as is the case for example in the processing of elastic synthetic fibres. The fabric receives its relative extensibility, which serves solely for the complete abutting of the bandage system against the body and/or the limbs, through the processing of the thread, having itself no or no substantial inherent elasticity, through the weaving, warp-knitting or knitting process. As a result of all these positive characteristics of the fabric, it can also be assumed that the bandage system according to the invention will be better accepted than conventional bandage systems by the patients.

In an advantageous embodiment of the invention, the width of the non-extensible fabric regions of the fabric is greater than the width of the fabric regions with relative extensibility. Thereby, an improved exterior support of the surrounded body areas is achieved as it were as a substitute of the supporting force of the skin which is lost temporarily or irretrievably in these body areas. In addition, constrictions at the body areas which are touched by the non-extensible fabric regions are prevented.

In an additional advantageous embodiment of the invention, in the region of joints the non-extensible fabric regions originate radially from the joint axis, and the fabric regions with relative extensibility radiate from the axis points arranged around the joint axis. The fabric regions with relative extensibility fill the wedge-shaped intermediate spaces between the non-extensible fabric regions. Thereby, in the region of joints, a secure abutting of the bandage system against the joint is achieved and a free, comfortable mobility of the limbs is guaranteed without slipping of the bandage system.

In a likewise advantageous embodiment of the invention, in the region outside the joints, the inner side of the non-extensible fabric regions is provided with small elevations of the same fabric, spaced apart from one another. Thereby, the drainage of the lymph is given an additional assistance.

In a particularly advantageous embodiment of the invention in this respect, these elevations run in the drainage direction of the territorial areas of the lymphatic system over the entire width of the non-extensible fabric regions. Thereby, the drainage of the lymph is directly given the natural direction.

The fabric regions of different extensibility can be achieved for example through different weaving techniques or the use of differently extensible materials within a working operation. Thus, in an advantageous embodiment of the invention, it is possible for example to produce the non-extensible fabric regions from materials which are worked into the extensible fabric which are not extensible by muscular strength, but are flexible. On the other hand, however, it is also possible to produce the non-extensible fabric regions by the working-in of threads which are not extensible by muscular force into the extensible fabric over the provided width of these fabric regions. This can take place advantageously during the production of the extensible fabric. In this production process, the finished fabric of the bandage system therefore arises as it were in one working operation. However, it is also possible to produce the non-extensible fabric regions in the required width at the desired positions into a finished extensible fabric subsequently by the working-in of threads which are non-extensible by muscular force.

Further advantages and advantageous embodiments of the invention can be seen from the following description, the claims and the drawings.

### BRIEF DESCRIPTION OF THE DRAWINGS

A preferred example embodiment of the bandage system according to the invention is illustrated in the form of a decongestion stocking is illustrated in the drawings and is explained more closely below. There are shown:

FIG. 1 a schematic illustration of a decongestion stocking according to the invention, applied to a leg,

FIG. 2 an enlarged cutout of the decongestion stocking in the region in the region of its upper end,

FIG. 3 an enlarged cutout of the decongestion stocking in the region of the knee,

FIG. 4 an enlarged cutout of the decongestion stocking in the region above the ankle joint of the leg,

FIG. 5 an enlarged cutout of the decongestion stocking in the region of the knee with a bent knee,

FIG. 6 a specific embodiment of the non-extensible fabric regions of the decongestion stocking and

FIG. 7 an enlarged cutout of the decongestion stocking in the region of its closure.

### DETAILED DESCRIPTION OF THE PREFERRED EMBODIMENTS

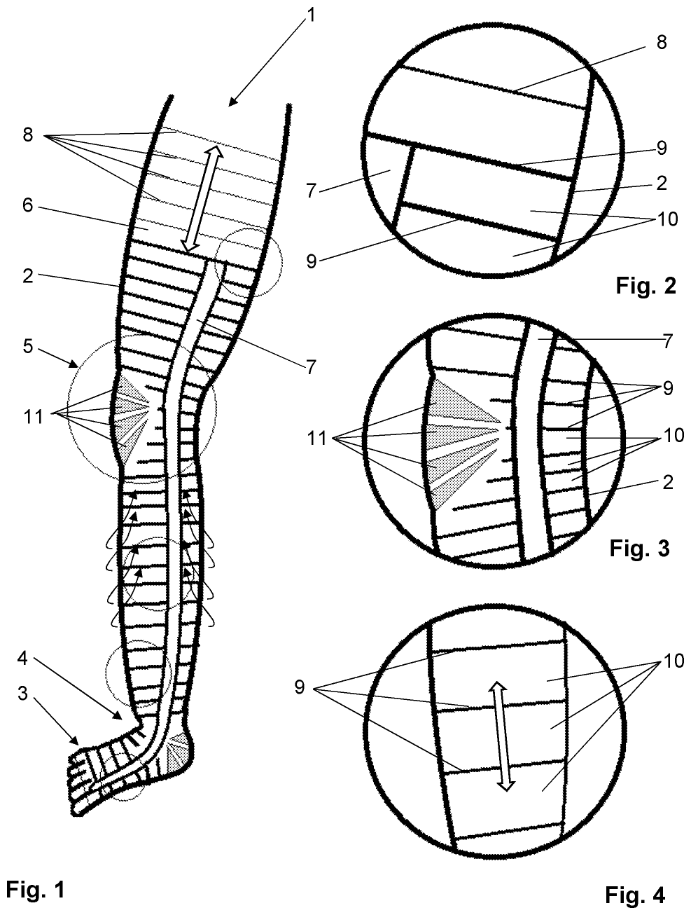

FIG. 1 shows a decongestion stocking 2 according to the invention, applied to a right leg 1, which stocking reaches from the metatarsophalangeal joint 3 of the foot over its ankle joint 4, the knee joint 5 up to below the end of the thigh stump 6. FIGS. 2 to 4 show enlarged cutouts at different locations of the decongestion stocking 2. On its inner side, the decongestion stocking 2 has a closable seam 7 reaching from the metatarsophalangeal joint 3 up to the thigh stump 6. In the region of the thigh stump 6, split lines 8 of the skin are indicated, which run transversely to the longitudinal direction or respectively working direction of the musculature, therefore substantially in circumferential direction of the leg 1. The working direction of the musculature is indicated in the region of the thigh stump 6 by a double arrow. FIG. 2 shows in addition an enlarged illustration from the region of the transition from the decongestion stocking 2 to the thigh stump 6. The curved arrows pointing from outside the decongestion stocking 2 obliquely upwards in the direction of the thigh stump indicate the drainage direction of the lymph fluid in the shank of the leg 1.

The decongestion stocking 2 consists of a fabric encasing the leg 1, which has alternately extensible fabric regions 9 and non-extensible fabric regions 10, wherein the non-extensible fabric regions 10, illustrated in white, are distinctly wider than the extensible fabric regions 9, drawn as thick solid lines. Both fabric regions 9, 10 run in the direction of the split lines 8 of the skin. In the detail cutout, illustrated in FIG. 4, above the ankle joint 4, the direction of the extensibility of the extensible fabric regions 9 is indicated by a double arrow.

To guarantee a comfortable mobility in the region of the joints, namely of the knee joint 5 and of the ankle joint 4, and a secure abutting of the bandage system in the region of the joints and in order to thereby prevent a slipping of the bandage system, additional extensible fabric regions 11, formed in a wedge-shaped manner, are provided between the non-extensible fabric regions 10 of the fabric of the joints 4, 5. These have an orientation adapted to the function of the joints in such a way that their tips, pointing towards the closure 7, do not originate from the central axis of the joints 4, 5, but rather from axis points 12 running around the latter, as can be seen from FIGS. 3 and 5.

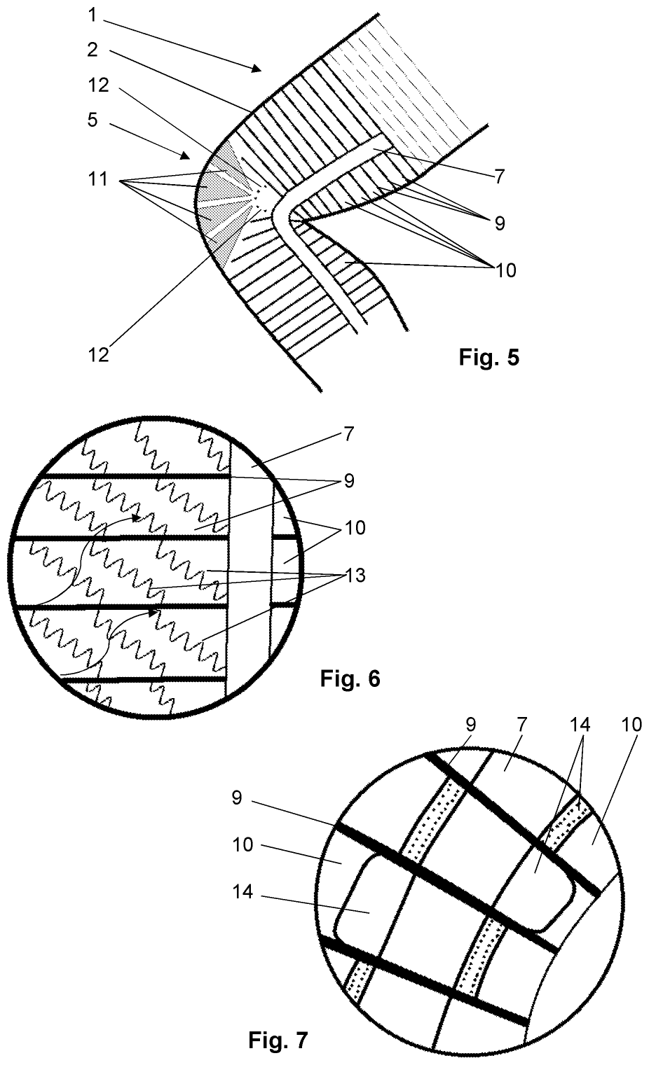

FIG. 6 shows an enlarged cutout of the decongestion stocking 2 according to the invention, in the region of the calf of the leg with a special feature, reinforcing its therapeutic effect, on the inner side of its non-extensible fabric regions. Elevations 13 of the same fabric are applied onto the inner side of the non-extensible fabric regions, which elevations run angularly to the fabric regions 9, 10 and become effective in the territorial areas in the drainage direction of the lymph. The drainage direction of the lymph is also indicated by the curved arrows in this illustration.

The closure of the seam 7 by way of example by means of hook-and-loop closures 14 is illustrated with the aid of the enlarged cutout of the decongestion stocking in the region of the metatarsophalangeal joint 3, shown in FIG. 7. It is self-evident that the closure of the seam 7 must take place over the entire length of the decongestion stocking 2.

All the features illustrated here can be essential to the invention both individually and also in any desired combination with one another.

### LIST OF REFERENCE NUMERALS

1 leg
2 decongestion stocking
3 metatarsophalangeal joint
4 ankle joint
5 knee joint
6 thigh stump
7 seam
8 split line
9 extensible fabric region
10 non-extensible fabric region
11 wedge-shaped extensible fabric region
12 axis point
13 elevations
14 hook-and-loop closure

1. A bandage system with decongestion function for the treatment of disorders of the lymphatic and/or venous system of the human body, consisting of a fabric which has alternately strip-shaped regions with relative extensibility and without extensibility and at least partially encases the human body and/or a limb,
characterized in that
the extension direction of the fabric regions of relative extensibility (9) is oriented longitudinally to the working direction of the musculature of the body or of the limbs,
that the fabric regions without extensibility (10) run transversely to the working direction of the musculature,
that the fabric regions of relative extensibility (9) and the fabric regions without extensibility (10) are arranged successively alternately in working direction of the musculature of the body or of the limbs and
that the bandage system is adjustable in its circumference.

2. The bandage system according to claim 1, characterized in that
the width of the non-extensible fabric regions (10) is greater than the width of the fabric regions (9) with relative extensibility.

3. The bandage system according to claim 1, characterized in that
in the region of joints, the non-extensible fabric regions (10) radiate from the joint axis and the fabric regions with relative extensibility from the axis points (12) arranged around the joint axis, and fill the wedge-shaped intermediate spaces between the non-extensible fabric regions (10).

4. The bandage system according to claim 1, characterized in that
in the region outside of joints, the inner side of the non-extensible fabric regions (10) is provided with elevations (13) of the same fabric, spaced apart from one another.

5. The bandage system according to claim 4, characterized in that
the elevations (13) in the drainage direction of the territorial areas of the lymphatic system run over the entire width of the non-extensible fabric regions (10).

6. The bandage system according to claim 5, characterized in that
the non-extensible fabric regions (10) are interrupted on their circumference.

7. The bandage system according to claim 6, characterized in that
the interruption (7) of the non-extensible fabric regions (10) in bandage systems for extremities is situated on the inner side thereof and in bandage systems for the torso on the front side thereof.

8. The bandage system according to claim 5, characterized in that
at the interruption (7) of the non-extensible fabric regions (10) an adjustable and/or re-adjustable closure system (14) is arranged.

9. The bandage system according to claim 1, characterized in that
the non-extensible fabric regions (10) consist of materials, not extensible by muscular force, but flexible, which are worked into extensible fabric.

10. The bandage system according to claim 1, characterized in that
the non-extensible fabric regions (10) consist of threads, non-extensible by muscular force, which are woven adjacent to one another into extensible fabric.
